SEQUENCE LISTING (11 sequences)

SEQ ID NO: 1 - DNA, 24 bp, Artificial Sequence
  Primer
tatgtgagtcaattccccaagtga

SEQ ID NO: 2 - DNA, 22 bp, Artificial Sequence
  Primer
atgatgaatgcatagatggatg

SEQ ID NO: 3 - DNA, 19 bp, Artificial Sequence
  Primer
ttgcagggaaaccacagtt

SEQ ID NO: 4 - DNA, 25 bp, Artificial Sequence
  Primer
tgaacatacatgtacatgtgtctgg

SEQ ID NO: 5 - DNA, 22 bp, Artificial Sequence
  Primer
cactgcagacggcatgaacttc

SEQ ID NO: 6 - DNA, 24 bp, Artificial Sequence
  Primer
gtggtattagtcaatgttctccag

SEQ ID NO: 7 - DNA, 19 bp, Artificial Sequence
  Primer
aatgtgtgtccttccaggc

SEQ ID NO: 8 - DNA, 20 bp, Artificial Sequence
  Primer
tccttggaataaattcccgg

SEQ ID NO: 9 - DNA, 25 bp, Artificial Sequence
  Primer
ttctctacatatttactgccaacac

SEQ ID NO: 10 - DNA, 23 bp, Artificial Sequence
  Primer
ccagaatcacatgagccaattcc

SEQ ID NO: 11 - DNA, 23 bp, Artificial Sequence
  Primer
cactgcagacggcatgaacttcc

(19) United States
(12) Patent Application Publication
(10) Pub. No.: US 2005/0123914 A1
(43) Pub. Date: Jun. 9, 2005

(54) Method of isolating cells and uses thereof

(76) Inventors: Mandy Katz, Victoria (AU); David Stephen Cram, Victoria (AU)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/488,855

(22) PCT Filed: Sep. 6, 2002
(86) PCT No.: PCT/AU02/01214

(30) Foreign Application Priority Data
Sep. 6, 2001 (AU) PR7499

(51) Int. Cl. C12Q 1/68; G01N 33/53; G01N 33/567; C12N 5/08

(52) U.S. Cl. 435/6; 435/7.2; 435/366

(57) ABSTRACT

The present invention relates to a non-invasive method of retrieving and identifying cells particularly fetal cells and trophoblastic cells. The invention includes methods for use of the cells for identifying chromosomal abnormalities and mutations particularly for prenatal diagnosis by performing genetic diagnosis for chromosomal and single gene disorders. The invention also includes methods of confirming cells of fetal origin.

The present invention relates to a non-invasive method of retrieving and identifying cells particularly fetal cells and trophoblastic cells. The invention includes methods for use of the cells for identifying chromosomal abnormalities and mutations particularly for prenatal diagnosis by performing genetic diagnosis for chromosomal and single gene disorders. The invention also includes methods of confirming cells of fetal origin.

### Introduction

Approximately 0.5% of couples are at high risk of conceiving a child with a genetic disorder. Such genetic disorders include Cystic Fibrosis, Huntington's Disease, Beta Thalassaemia and Myotonic Dystrophy. For example, in Australia, 1 in 25 of the population is a carrier of a Cystic Fibrosis mutation and thus newborn screening for Cystic Fibrosis has recently been implemented to monitor all births.

In addition to single gene disorders chromosomal abnormalities are the most common genetic disorders seen in spontaneous miscarriages and newborn babies. Trisomies involving chromosomes 21, 18, 13, X and Y are the largest group with trisomy 21 or Down syndrome being the most frequent, occurring in approximately one in every 700 live births. Trisomies 13 and 18 are the only other autosomal trisomies that reach full term with a characteristic malformation syndrome leading to death during the immediate postnatal period. The remaining liveborn trisomic individuals have an additional sex chromosome, XXY, XYY or XXX. Prenatal diagnosis is primarily undertaken in an attempt to detect chromosomal abnormalities in the fetus, particularly Down syndrome. Down syndrome is the most important genetic cause of mental retardation in humans and is also associated with a high risk of congenital heart disease and leukaemia.

Prenatal diagnosis is performed to detect either single gene disorders or chromosomal abnormalities in the fetus during pregnancy. Currently, prenatal diagnosis involves an invasive procedure either in the form of chorionic villous sampling (CVS) (10-12 weeks) or amniocentesis (14-16 weeks) to identify potential chromosomal aneuploidies in the fetus. Both these procedures are associated with a risk of miscarriage (1-2%). Therefore, prenatal testing is only offered to women perceived to be at increased risk, including those of advanced maternal age (>35 years), those with abnormal maternal serum screening or those who have had a previous fetal chromosomal abnormality.

Prenatal diagnosis is usually performed by an invasive method to sample chorionic or amniotic cells. These methods of sampling ensure fetal cells of the current fetus are also tested. Samples obtained from other sources such as the blood cannot ensure that the fetal cell so identified, may be derived from the current fetus or of a recently miscarried fetus because such cells can persist in the circulation for several years. Once the fetal cell is obtained cytogenetic techniques are used to identify chromosomal abnormalities. Such procedures are, lengthy and require a high level of technical expertise. Further, results generally are not available to the patient for up to three weeks.

Therefore a rapid, non-invasive diagnostic technique and preferably one which ensures the testing of a current fetus would significantly benefit all pregnant women of high or low genetic risk. A diagnosis within 24 hours would give them piece of mind and the opportunity to make an earlier decision regarding therapeutic abortion in the first trimester of their pregnancy.

Hence, there is a need for a rapid and non-invasive diagnostic test for pregnant women to identify substantially intact fetal cells and diagnose common fetal chromosomal aneuploidies such as Down syndrome from their current pregnancy as well as other genetic and single gene disorders.

Accordingly, it is an aspect of the present invention to overcome or at least alleviate some of the problems of the prior art and improve genetic testing for pregnant woman.

### SUMMARY OF THE INVENTION

In a first aspect of the present invention there is provided a method of retrieving cells from a cervical mucus sample, said method comprising:
obtaining a cervical mucus sample;
treating the sample with a collagenase and a protease to disassociate cells from the cervical mucus sample; and
retrieving disassociated cells from the sample.

It is preferred that the present method retrieves substantially intact cells that have substantially maintained their cell membrane integrity which allows for reliable identification such as through antibody testing.

In a preferred embodiment, the cervical mucus sample is further treated with a mucolytic agent prior to being treated with a collagenase and a protease to disassociate the cells. It has been found that by treating in this combined manner, there is a better yield of suspended single cells from the cervical mucus sample.

The mucus sample is further treated with an enzyme mixture to break down the mucus. Ideally, the mixture maintains the integrity of the cells to preserve cellular membranes to facilitate identification of the cells either as fetal or maternal. Hence the enzymes have been selected in combination, which do not substantially effect the cell.

Applicants have found that the combination of protease with collagenase and preferably with the mucolytic agent, successfully releases cells in a form that allows their identification and use for subsequent diagnostic purposes. In yet another aspect of the present invention there is provided a method of retrieving a fetal cell from a cervical mucus sample, said method comprising:
obtaining disassociated cells as described above;
treating the cells with fetal-specific antibodies;
identifying cells that have bound to the antibodies; and
retrieving the identified fetal cells.

In another aspect of the present invention there is provided a method of identifying a fetal cell, said method comprising:
obtaining disassociated cells from a cervical mucus sample as described above;
treating the cells with fetal-specific antibodies; and
identifying cells that have bound to the antibodies.

In another aspect of the present invention there is provided a method of identifying a chromosome aneuploidy in a chromosome of a fetal cell said method comprising:
obtaining a fetal cell;
identifying at least three polymorphic microsatellite markers on the chromosome; and
determining an allelic profile of at least three (3) polymorphic microsatellite markers.

In another aspect of the present invention there is provided a method of prenatal diagnosis, said method comprising:
obtaining a fetal cell from a cervical mucus sample as described herein;
identifying at least three (3) polymorphic microsatellite markers on the chromosome characteristic of the fetal cell;
determining an allelic profile of the at least three (3) polymorphic microsatellite markers; and
correlating the allelic profile with a condition for prenatal diagnosis.

In a preferred aspect, there is provided a method of diagnosing Down syndrome said method comprising identifying a chromosome aneuploidy by a method comprising:
obtaining a fetal cell;
identifying at least three polymorphic microsatellite markers on the chromosome;
determining an allelic profile of the at least three (3) polymorphic microsatellite markers; and

determining a trisomy of chomosome 21.

In another aspect of the present invention there is provided a method of confirming fetal origin of a cell from a cervical mucus sample from an individual, said method including:
obtaining a fetal cell and a maternal cell from the same individual;
selecting at least three (3) polymorphic microsatellite markers characteristic of either the fetal or maternal cell; and
determining an allelic profile of the at least three (3) polymorphic microsatellite markers on the fetal cell and the maternal cell.

### FIGURES

FIG. 1 shows a DNA fingerprint of a single human buccal cell from a male subject with Down syndrome. Microsatellite markers D21S1413, D21S11 and D21S1442 show tri-allelic patterns, while D21S1437 and D21S1411 show di-allelic double dosage patterns with the expected 1:2 allelic ratio.

FIG. 2 shows a DNA fingerprint of a single human buccal cell from a diploid subject. In this octaplex DNA fingerprinting system there are two microsatellite markers for each of the following chromosomes, X, 13, 18 and 21 each displaying a diploid allelic ratio.

FIG. 3 shows a DNA fingerprint of a single human buccal cell from a diploid individual who is a carrier of the common Cystic Fibrosis deltaF508 mutation. In this DNA fingerprint there are four microsatellite markers for chromosome 21 combined with mutation detection for Cystic Fibrosis deltaF508.

### DETAILED DESCRIPTION

In a first aspect of the present invention, there is provided a method of retrieving cells from a cervical mucus sample, said method comprising;
obtaining a cervical mucus sample;
treating the sample with a collagenase and a protease to disassociate cells from the cervical mucus sample; and
retrieving disassociated cells from the sample.

The present invention provides a method to liberate cells, both maternal and fetal, from cervical mucus samples. These cells may be trapped in the complex mucus structures and attempts have previously been made to release these cells. However, the cells have not been successfully released previously, and if they were they remained in clumps or their cell membrane integrity was destroyed thereby reducing their effectiveness for subsequent use such as prenatal diagnosis or effective identification.

Accordingly, it is preferred that the present method retrieves substantially intact cells that have substantially maintained their cell membrane integrity which allows for reliable identification such as through antibody testing.

To correctly diagnose a genetic disorder of a fetus, the fetal cell is ideally used. However, it has been a problem to obtain reliable isolation and identification of a fetal cell for such uses. The cervical mucus provides a source of these cells but the problem remained to effectively isolate a fetal cell from the mucus, that contains principally maternal cells, then maintain its integrity for identification and diagnostic purposes.

Non-invasive methods of testing the fetus would reduce the incidence of miscarriage and fetal death. Fetal cells shed into the lower uterine pole and cervical mucus of the mother have provided a potential source of fetal cells. However, associated with this source is the problem of isolating the fetal cells for further identification and diagnosis. Until now, it has been difficult to isolate these scarce cells from the surrounding mucus plug. Even when the cells are liberated from the mucus, the problem remained to isolate and identify the rare fetal cells from maternal cells. Hence a positive identification of these scarce cells from the majority of maternal cells was required before subsequent single cell molecular diagnosis for any genetic disorders could be conducted.

Previous studies suggest that these cells originating from the current fetus only appear in a narrow window of 7 to 13 weeks gestation.

Nucleated red blood cells of fetal origin have been found in the maternal circulation during the first and second trimesters of pregnancy, albeit at a frequency of approximately 1 in a million. Several groups have used different forms of cell sorting in an attempt to isolate these rare fetal nucleated red blood cells; however with limited success. In addition, studies have shown the perseverance of fetal cells in the maternal circulation post delivery, and thus, could confound the diagnosis of the current fetus. The use of fetal-specific antigens on the surface of fetal red blood cells and other fetal cells, combined with micromanipulation techniques, has to date, been most promising for identifying fetal cells.

The term “intact” cells means a cell which maintains cell membrane integrity. The cells ideally have not lost intracellular content so as to allow for further identification by the use of nucleic acids including DNA, RNA and mRNA.

The present method provides a means to obtain cells preferably intact cells from cervical mucus which provides a basis for a non-invasive test of the fetus. Once the cells are liberated from the mucus plug, they may be further identified as fetal or maternal thereby providing a source of fetal cells for genetic testing.

The cells in the sample include cells from the endocervical canal that have shed from the fetus and migrated to the cervix. These cells have been found in the endocervical canal during the first trimester (approximately 7-13 weeks) of pregnancy. The cells may be of fetal or maternal origin.

It is hypothesised that these cells have shed from the regressing chorionic villis into the lower uterine pole and cervical mucus. Fetal cells can be retrieved along with maternal cells in a non-invasive method, similar to a pap smear preferably by aspiration of the mucus from the endocervical canal and the lower uterine pole. Previous studies have shown that these fetal cells occur in 50-90% of transcervical samples; the variability in frequency has been due to the sampling technique, skill of the operator and the inability to definitively distinguish fetal from maternal cells. It has been reported in the literature that the collection of transcervical cells is safe and efficient. Preliminary studies performed on pregnant women prior to a CVS suggest that this procedure does not increase the risks of infection or spontaneous abortion. In studies reported to date involving more than 200 women, cervical samples were aspirated during ongoing pregnancies and these procedures have not had any deleterious affects on the health of the mother or fetus. Fetal origin has only been confirmed in some samples by the presence of paternally inherited microsatellite markers after PCR of a large number of transcervical cells. Maternal cell contamination will interfere with prenatal diagnosis.

The cervical mucus sample may be obtained at any stage of pregnancy. Preferably the sample is obtained during the first and second trimester of pregnancy. Ideally the sample is obtained at a stage when a decision can be made for the well-being of the fetus and preferably within a period where an opportunity to make an early decision regarding therapeutic abortion can be made. Preferably the sample is obtained up to 14 weeks of the pregnancy. More preferably, the sample is obtained in the first trimester of pregnancy.

In a preferred embodiment, the cervical mucus sample is further treated with a mucolytic agent prior to being treated with a collagenase and a protease to disassociate the cells. It has been found that by treating in this combined manner, there is a better yield of the cells from the cervical mucus sample.

Suitable mucolytic agents may be selected from the group including N-acetyl-L-cysteine, DTT, trypsin and trypsin/EDTA. Preferably, the mucolytic agent is N-acetyl-L-cysteine.

The sample is preferably treated with the mucolytic agent prior to treatment with the enzymes. However, this step may also be conducted in combination with the enzyme treatment with a collagenase and a protease. The combination of treatments results in a synergistic effect of mucus breakdown thereby facilitating the release of cells. The combined effect of the mucolytic agent and enzymes (collagenase and protease) is more effective than the separate and summed effects of mucolytic agent alone and enzymatic treatment alone.

Preferably, the sample is treated with 2-20 mg/ml of N-acetyl-L-cysteine. More preferably, a final concentration of 10 mg/ml is used.

The sample is treated for a period sufficient to resolve the mucus and generally to reduce the viscosity of the mucus by dissociating the mucus plug into small globules. More preferably the sample is treated at approximately 37” C. for a period of 30 to 60 minutes. Most preferably the sample is treated for 45 minutes preferably with gentle agitation.

The mucus sample is further treated with an enzyme mixture to break down the mucus. Ideally, the mixture maintains the integrity of the cells to preserve cellular membranes to facilitate identification of the cells either as fetal or maternal. Hence the enzymes have been selected in combination which do not substantially effect the cell.

The use of enzymes such as proteases are generally avoided particularly if the integrity of the cell membrane is to be maintained. However, applicants have found that the combination of a protease with a collagenase and preferably with the mucolytic agent, successfully releases cells in a form that allows their identification and use for subsequent diagnostic purposes.

The collagenase and protease may be used singularly or in combination. However, it is preferred that both enzymes are used simultaneously to treat the mucus sample. It is also desirable to prepare the enzymes in a mixture for the treatment of the mucus sample so that simultaneous treatment of the mucus sample is achieved.

Preferably, the concentration of the enzyme is sufficient to substantially break down the mucus. The concentration will preferably be high enough to break down the mucus in at least one or two treatments.

The cervical mucus sample is treated with a collagenase and a protease. Any collagenase type or protease type familiar to the skilled addressee may be used.

Commercially available mixes of enzymes such as liberase blendzyme may be used to complement the collagenase and protease treatment. Liberase blendzyme is a combination of collagenase isoform I and II and thermolysin and can be obtained from Roche. A suitable concentration of the enzyme mixture is approximately liberase blendzyme (0.5-10 WU/ml) collagenase and dispase (0.1-1.5 mg/ml).

However the invention is not limited to these specific concentrations. Manipulation of the concentration and incubation time could provide better liberation of the cells in some mucus samples.

The disassociated cells will comprise maternal and fetal cells and it is from this cell mixture that the fetal cells may be further identified and isolated for use in prenatal diagnostic tests or for other uses which require the isolated cells or fetal cells.

The disassociated cells may be retrieved from the sample by any method available to the skilled addressee including centrifugation after washing with suitable buffers and salines. Retrieval or removal of the cells involves the separation of the cells from the supernatant. Once retrieved, the cells may be used for further identification into maternal and fetal cells.

In yet another aspect of the present invention there is provided a method of retrieving a fetal cell from a cervical mucus sample, said method comprising:
obtaining disassociated cells as described above;
treating the cells with fetal-specific antibodies;
identifying cells that have bound to the antibodies; and
retrieving the identified fetal cells.

The fetal cells may be retrieved from the cervical mucus sample after preparing a sample of disassociated cells as described above. The disassociated cells from the cervical mucus sample are a mix of fetal and maternal cells. The mixture of the cells may then undergo identification to identify cells of fetal origin.

The fetal cell of the present invention may be identified and isolated using techniques well known in the art. These techniques include, but are not limited to, immunohistochemistry including the use of antibodies to label a cell and hence identify it as being of fetal origin. These techniques also include the use of primary and secondary antibodies to identify the cell as being of fetal origin of the first trimester. Preferably the antibodies bind to fetal-specific antigens and could be IgG, IgM and monoclonal. Preferably the fetal-specific antigens are located on the surface of the fetal cell. Once bound, the fetal cell is processed to separate the ‘labelled’ cells from those that lack the label. Labelling of the cell may include the further use of a secondary antibody that binds to the primary antibody. Examples of secondary antibodies include rabbit anti-mouse fluorescein isothiocyanate isomer I (FITC) which will bind to a mouse derived primary antibody. However, the primary antibody may be suitable to identify and isolate the cell in the absence of the secondary antibody. Suitable secondary antibodies may be determined by the skilled addressee by consideration of the primary antibody and reacting the secondary antibody to the primary antibody.

The fetal cell is identified by fetal specific antibodies. Any presently available fetal specific antibodies can be used once the cells are separated from the mucus of the cervical sample. Preferably, the fetal specific antibodies are specific for the first trimester of pregnancy. Most preferably, the antibodies include antibodies specific for syncytiotrophoblasts, villous cytotrophoblasts and cytotrophoblast cell columns.

Other suitable fetal specific antibodies are those described in Sunderland, C. A et al (1981) “Monoclonal Antibodies to human syncytiotrophoblast”, Immunology 43(3):541-6 and in Griffith-Jones, M. D. et al (1992) “Detection of fetal DNA in trans-cervical swabs from first trimester pregnancies by gene amplification: A new route to prenatal diagnosis?”, British Journal of Obstetrics and Gynecology, 99(6):508-11. Specifically, these antibodies are listed as NDOG1, NDOG5 and FT1.41.1. NDOG1 stains the syncytiotrophoblasts, NDOG5 the syncytiotrophoblasts and cytotrophoblast cell columns and FT1.41.1 the syncytiotrophoblasts and villous cytotrophoblasts of first trimester pregnancy. None of these antibodies are reactive to maternal endometrium or cervical tissue.

These antibodies may be used singularly or in combination. These antibodies may be subjected to the cells separately or simultaneously, providing the cells are allowed to react and bind to the antibodies. Preferably, they are used as an antibody mix to identify the fetal cells. It has been found by the applicants that these antibodies specifically bind to cell membranes of fetal cells.

It is preferred that the fetal specific antibodies are specific for all fetal cell types. Due to the heterogeneity of the fetal cells arising in the cervical mucus sample, it is desirable to use a mixture of fetal specific antibodies to detect all types of fetal cells. Where one antibody is used, other fetal cell types may be missed. Antibodies may be selected by knowing the stage of pregnancy and hence a particular cell type may be predicted. Accordingly, antibodies specific to that predicted cell type may be preferentially used alone or in combination.

The antibodies may include a label to facilitate identification. The term “label” when used herein refers to a compound or composition which is conjugated or fused directly or indirectly to a reagent such as a nucleic acid probe or an antibody and facilitates detection of the reagent to which it is conjugated or fused. The label itself may be detectable (e.g. radioisotope labels or fluorescent labels) or, in the case of an enzymatic label, may catalyze chemical alteration of a substrate compound or composition which is detectable.

Fetal cells that have been labelled with an antibody may be identified and/or separated using techniques well known in the art including fluorescent activated cell sorting (FACS), magnetic bead separation techniques, micromanipulation techniques, laser capture and fluoroimmunohistochemistry for either the negative selection of maternal cells or the positive selection of fetal cells. Preferably the fetal cells are identified and/or separated using fluoroimmunohistochemistry for either the negative selection of maternal cells or the positive selection of fetal cells. For example, fetal cells that have been labelled using fluoroimmunohistochemistry may be morphologically identified under a fluorescent microscope and the cells isolated using micromanipulation techniques using, for example, pulled glass pipettes or micromanipulators.

Once the cell is identified, it may then be isolated or retrieved by methods available to the skilled addressee. For instance, if the cells are fluorescently labeled they may be isolated by laser capture or sorted by FACS analysis. However, other methods may be used depending on the methods of identification of the cells identified by the antibodies.

In another aspect of the present invention, there is provided a fetal cell retrieved by the methods described herein.

In another aspect of the present invention there is provided a method of identifying a fetal cell, said method comprising:
obtaining disassociated cells from a cervical mucus sample as described above;
treating the cells with fetal specific antibodies; and
identifying cells that have bound to the antibodies.

Successful identification of the fetal cell from a cervical mucus sample is acheived by obtaining a cell suspension from the cervical mucus sample. The cells are preferably intact which allows the antibodies to react to the substantially intact cell membrane.

In another aspect of the present invention there is provided a method of identifying a fetal cell said method comprising:
obtaining a cell sample;
treating the cell sample with an antibody selected from the group including NDOG1, NDOG5 and FT1.41.1 as herein described or equivalent thereof; and
identifying cells that have bound to the antibodies.

As described above, the fetal cells may be identified using a specific cocktail of antibodies reactive only to fetal cells. These antibodies namely NDOG1, NDOG5 and FT1.41.1 are specific for fetal cells. They may be used singularly or in combination and they may be added separately or simultaneously.

The term “equivalent thereof” as it applies to the antibodies NDOG1, NDOG3 and FT1.41.1 and as used herein means an equivalent antibody which behaves in a similar manner and which has a similar specificity to any one of the listed antibodies. For instance, other antibodies may be generated by determining the target sites of NDOG1, NDOG5 and FT1.41.1 and using methods familiar to the skilled addressee such as those for generating monoclonal and polyclonal antibodies.

Preferably, the antibodies are labelled in a manner as described above. Fluorescent labelling is most preferred.

Once fetal cells are identified and retrieved preferably from the non-invasive source such as the cervical mucus plug, the cells may be used in any manner including:
(a) multiplex FL-PCR for fetal identification, chromosomal aneuploidy and single gene diagnosis;
(b) WGA, hybridisation and microarray analysis with SNP's for fetal identification and probes for single gene disorders and chromosome aneuploidy; or
(c) extraction of mRNA, cDNA libraries, hybridisation and gene expression microarray analysis.

These techniques may be used to characterise the fetal cell to identify any biochemical, metabolic or genetic disorders of the fetal cell. The isolated fetal cell may be used to identify all types of abnormalities including fetal abnormalities that can be determined in any cell type. Any cellular analysis can be performed on the fetal cell once it is isolated and identified from the cervical mucus sample.

Microarrays may also be used to confirm fetal origin, diagnosis of single gene disorders and chromosomal abnormalities. On a single microarray it is possible to identify fetal cells using single nucleotide polymorphisms (SNP's), as well as single gene disorders and chromosomal abnormalities. With this method the isolated and identified single fetal cell may undergo whole genome amplification (WGA) by either primer extension preamplification PCR(PEP-PCR), degenerate oligonucleotide primed PCR (DOP-PCR), linker adapter-PCR or MSD (multiple strand displacement) WGA. Fluorescently labelled product from WGA can be hybridised to the microarray platform and laser scanning of bound fluorescence will confirm fetal origin and diagnosis of chromosome aneuploidy of all 23 pairs of human chromosomes and identify any specific single gene defects.

In yet another aspect of the present invention there is provided a composition when used for identifying fetal cells, said composition comprising antibodies NDOG1, NDOG5 and FT1.41.1.

In another aspect of the present invention there is provided a method of identifying a chromosome aneuploidy in a chromosome of a fetal cell said method comprising:
obtaining a fetal cell;
identifying at least three polymorphic microsatellite markers on the chromosome; and
determining an allelic profile of at least three (3) polymorphic microsatellite markers.

This aspect of the present invention relates to a method of identifying a chromosome aneuploidy in a chromosome of a fetal cell. “Chromosome aneuploidy in a chromosome” as used herein includes a chromosome missing or having an extra copy or part of a chromosome as compared to the normal native karyotype of a subject and includes deletion, addition and translocation, which causes monosomy or trisomy at particular sites. Preferably the aneuploidy is selected from the group including trisomy and monosomy of autosomes, and monosomy, disomy and trisomy of sex chromosomes.

Preferably the fetal cell is obtained from a cervical mucus sample as described above. Preferably, the sample is from a pregnant woman. However, this invention does not exclude obtaining fetal cells from a woman who has miscarried.

Invasive methods of obtaining fetal cells such as chorionic villous sampling (CVS) or amniocentesis give rise to chorionic villus samples, aminocytes, fetal tissues and cord blood can provide fetal cells. However, non-invasive methods for obtaining fetal cells from cervical mucus samples is preferred. Any type of fetal cell at any stage may be used.

The present invention includes the use of polymorphic microsatellite markers specific for a nucleic acid. The nucleic acid of the present invention may be DNA, preferably chromosomal DNA. Preferably the polymorphic microsatellite markers are located on the same chromosome.

Preferably the polymorphic microsatellite markers are selected based on high heterozygosity, broad distribution of alleles, high probability of producing a tri-allelic pattern and specificity to the indicated chromosome. For example see chromosome 21 tetranucleotide microsatellite markers for diagnosis of Down syndrome listed in Table 2 and other tetranucleotide microsatellite markers diagnostic for other syndromes listed in Table 3.

The polymorphic markers selected will be useful for identifying various patterns of aneuploidy including trisomy and monosomy of autosomes, and monosomy, disomy and trisomy of sex chromosomes. The broad distribution of allelic sizes is preferred for successful genetic analysis since such range of allelic sizes provides an allelic pattern diagnostic of aneuploidy, particularly trisomy, disomy or monosomy. The markers are also selected so that each marker has a distinct allelic profile in the DNA fingerprint which does not overlap with other markers.

Trisomies are the most common chromosome abnormalities often seen in miscarriages and stillbirths, with trisomy in chromosomes 21, 18, and 13, and disomy of X and Y being the largest group. Trisomy 21 or Down syndrome is the most common autosomal chromosomal abnormality that reaches term.

The present invention requires at least three polymorphic microsatellite markers on the chromosome to allow identification of a chromosome aneuploidy.

Amplification of less than three polymorphic markers provides spurious results due to several reasons. Problems associated with DNA fingerprinting by multiplex fluorescent polymerase chain reaction (FL-PCR) on a limited template include total amplification failure, the possibility of parental homozygosity (each parent having two copies of the same allele), allele dropout (ADO) (the total amplification failure of one allele in the first few cycles of the PCR to such an extent that only one allele is detectable) and preferential amplification (PA) (the under-representation of one allele resulting in a distortion from the expected 1:1 di-allelic ratio). Hence, a minimum of three highly polymorphic microsatellite markers per chromosome is required for diagnosis of an aneuploid cell.

For improved accuracy, the number of microsatellite markers may be increased. The method requires at least three (3) markers. However, five (5) microsatellite markers are preferred. With the inclusion of at least three (3) microsatellite markers, allelic dropout (ADO) and preferential amplification does not interfere as much with the result since if one locus marker is affected, others remain for the definitive diagnosis. Preferably five polymorphic markers are amplified.

Throughout the description and claims of this specification, the word “comprise” and variations of the word, such as “comprising” and “comprises”, is not intended to exclude other additives, components, integers or steps.

The allelic profile provides a means for identifying aneuploidy and fetal origin. A ratio of the various alleles identified by the polymorphic microsatellite markers provides an allelic pattern identifiable as anyone of the various forms of aneuploidy including, but not limited to, trisomy, disomy and monosomy.

Various means are available to detect alleles by the polymorphic microsatellite markers. Other methods include restriction fragment polymorphisms (RFLP's), single nucleotide polymorphisms (SNP's) and microarrays.

In a preferred embodiment, the allelic profile is determined by amplification of polymorphic markers to generate an allelic profile. An allelic profile may provide a specific indication of aneuploidy including but not limited to monosomy, disomy or trisomy. For example, since the amount of DNA produced in FL-PCR amplification is estimated as being proportional to the quantity of the initial target sequence, allelic ratios for any particular loci can be calculated from the final fluorescent yield (the amount of PCR product from the first allele divided by the amount of product of the second allele). Accordingly, a disomy can be defined by an expected bi-allelic ratio of 1:1, whereas a trisomy can be defined as a tri-allelic pattern and an expected ratio of 1:1:1. The diagnosis of a monosomy may require all 5 microsatellite markers to display a single allele, while a trisomy could be definitively diagnosed by at least one tri-allelic pattern of one marker.

The term “amplifying” as used herein includes any of a variety of methods known to those of skill in the art that increase the number of copies of a nucleic acid or portions thereof. Nucleic acid amplification can be accomplished by any of the various nucleic acid amplification methods known in the art, including the polymerase chain reaction (PCR). Various forms of PCR are encompassed within the scope of the present invention including multiplex FL-PCR. A variety of amplification methods are known in the art and are described in “The polymerase chain reaction”, Baumforth et al., Journal of Clinical Pathology: Molecular Pathology 1999, (52): 1-10.

Amplified nucleic acid may be analysed, and a profile generated, using techniques well known to those of skill in the art including polyacrylamide gel electrophoresis (PAGE), preferably using a denaturing gel. Analysis may be performed using automated or manual procedures, for example automated analysis may include use of an ABI Prism 377 DNA Sequencer and associated Genescan 672 software (Applied Biosystems Australia). Other automated analysis includes the ABI Prism 3100 Genetic Analyzer and denaturing high phase liquid chromatography (DHPLC).

The term “primers” as used herein includes short nucleic acids, preferably DNA oligonucleotides 15 nucleotides or more in length, that can be annealed to, for example, a complementary target DNA strand by nucleic acid hybridization to form a hybrid between the primer and the target DNA strand, then extended along the target DNA strand by a polymerase, preferably a thermostable DNA polymerase. Primer pairs can be used to amplify a nucleic acid, e.g., by PCR or by other nucleic acid amplification methods well known in the art. PCR-primer pairs can be derived from the sequence of a nucleic acid and designed according to the following criteria, approximately 50% GC content, 18-24 base pairs in length, minimal primer-dimer formation and self annealing, 2 G or C bases at the 3′ end of the primer, forward and reverse primers to be the same length (± one nucleotide), no more than three repeated bases in a row and the size of the PCR product to be between 100-400 nucleotides in length.

Methods for preparing and using primers are described, for example, in Sambrook et al. Molecular Cloning: A Laboratory manual, 2^{nd} ed., vol. 1-3, ed. Sambrook et al., Cold Spring Harbor Laboratory Press, Cold Spring Harbor, N.Y., USA 1989; Current Protocols in Molecular Biology, ed. Asubel et al., Greene Publishing and Wiley-Interscience, NY, USA 1987.

Most preferably the present method includes determining the allelic profile of the polymorphic microsatellite markers by DNA fingerprinting.

In another aspect of the present invention there is provided a method of prenatal diagnosis, said method comprising:
obtaining a fetal cell from a cervical mucus sample as described herein;
identifying at least three (3) polymorphic microsatellite markers on the chromosome characteristic of the fetal cell;
determining an allelic profile of the at least three (3) polymorphic microsatellite markers; and
correlating the allelic profile with a condition of prenatal diagnosis.

The term “prenatal diagnosis” as used herein includes determining the presence of a genetic mutation or any biochemical or metabolic identification in a fetal cell. The prenatal diagnosis is intended to identify all types of fetal abnormalities. The genetic mutation includes, but is not limited to chromosomal aneuploidies, point mutations, translocations, trinucleotide repeat expansions, inversions, polymorphisms, insertions and deletions. The genetic mutation may cause a gene disorder, for example cystic fibrosis, beta-thalassaemia, Huntington's Disease, Fragile X, Myotonic Dystrophy, Duchenne Muscular Dystrophy or Sickle Cell Anaemia.

Prenatal diagnosis may include genetic disorders which occur due to abnormalities in the chromosome. Chromosome abnormalities involving chromosomes 21, 18, 13, X and Y are the most frequent and are found in live births. Other aneuploidies are generally lost prior to implantation or early in the first trimester however with an earlier non-invasive method diagnosis of aneuploidies of all 23 pairs of chromosomes can be achieved with multiplex FL-PCR or microarrays. Genetic disorders including, but not limited to Turners syndrome (XO), Klinefelter's syndrome (XXY), XXX females and XYY males, Triploidy (69, XXX or XXY or XYY), Patau's syndrome (trisomy 13) and Edward's syndrome (trisomy 18). Down syndrome (trisomy 21) may also be detected by the present method. Preferably the prenatal diagnosis can detect chromosomal abnormalities in the form of aneuploidies. Prenatal diagnosis may also include single gene disorders caused by mutation in specific genes. The most common disorder is Cystic Fibrosis. Cystic Fibrosis is observed in 1 in 2,500 births and 1 in 25 individuals are carriers of this autosomal recessive condition.

In a preferred aspect, there is provided a method of diagnosing Down syndrome said method comprising identifying a chromosome aneuploidy by a method comprising:
obtaining a fetal cell;
identifying at least three polymorphic microsatellite markers on the chromosome;
determining an allelic profile of the at least three (3) polymorphic microsatellite markers; and
determining a trisomy of chromosome 21.

Currently, there are a series of non-diagnostic serum screening tests that are offered to pregnant women who are less than 35 years of age for the detection of a Down syndrome fetus. These tests involve the measurement of a range of variables that have been identified to correlate with a Down syndrome fetus including, the amount of fluid accumulation behind the fetal neck (nuchal thickness) measured at a 12 week ultrasound, the concentration of free-beta hCG and pregnancy associated plasma protein-A (PAPP-A) in the maternal blood in the first trimester and the concentration of free-beta hCG and alpha-fetoprotein (AFP) in the maternal blood in the second trimester. From combined test results, a patient's specific risk can be calculated with a 80-90% detection efficiency and a 5-10% false positive rate. However, these screening tests are non-diagnostic and a negative result does not indicate the absence of Down syndrome in the fetus. Currently nearly 80% of Down syndrome live births are to mothers under 35 years of age.

The fetal cells may be obtained from any source as described above. Preferably, the cells are fetal cells obtained from the transcervical swabs or aspirations of the cervical canal of a pregnant female. This is ideal for a non-invasive method of diagnosis.

In the present method, a trisomy of chromosome 21 is indicative of Down syndrome.

The method is performed with at least three (3) polymorphic microsatellite markers. However, it is preferred that at least five (5) markers are used. Tetranucleotide microsatellite markers on chromosome 21 that are highly heterozygous and which have a broad distribution of allelic sizes is particularly preferred for this method.

For Down syndrome, selection of markers which generate a distinct allelic profile or pattern which do not have any fluorescent or size overlap are preferred. See Table 2 for preferred diagnosis of Down syndrome.

Whilst the present method requires at least three (3) microsatellite markers, preferably selected from Table 2, it is most preferred that five (5) markers are used to diagnose Down syndrome.

With the inclusion of five (5) microsatellite markers in this system, allelic dropout and preferential amplification does not interfere with the result since if one locus marker was affected, there are four remaining for a definitive diagnosis.

The method of determining an allelic profile may be by any method which generates a pattern indicative to the alleles represented by the polymorphic microsatellite markers. Preferably, the allelic profile is determined by DNA amplification of the markers, preferably using PCR, more preferably using FL-PCR.

Methods of analysis of the PCR products are described herein.

Once a profile is established, the aneuploidy can be identified as described above with respect to the amount of DNA, their respective allelic ratios and sizes determined therefrom see FIG. 2 for Down syndrome and FIG. 3 for aneuploidies of chromosomes 13, 18, 21 and X.

In another aspect of the present invention, there is provided a microsatellite marker for use in a method for diagnosing Down syndrome, said marker having a forward primer sequence including a sequence selected from any one of:

tatgtgagtcaattccccaagtga;
atgatgaatgcatagatggatg;
ttgcagggaaaccacagtt;
tgaacatacatgtacatgtgtctgg;
or
cactgcagacggcatgaacttc.

In yet another aspect of the present invention, there is provided a microsatellite marker for use in a method for diagnosing Down syndrome, said marker having a reverse primer sequence including a sequence selected from any one of:

gttgtattagtcaatgttctccag;
aatgtgtgtccttccaggc;
tccttggaataaattcccgg;
ttctctacatatttactgccaacac;
or
ccagaatcacatgagccaattcc.

In a preferred embodiment of the present invention, there is provided a microsatellite marker for use in a method for diagnosing Down syndrome, wherein said marker is any one marker selected from the group described in Table 2. Any one of these markers may be used in combination with at least two (2) other suitable markers for diagnosing a trisomy 21 according to the methods described herein. In addition, primer sequences can be redesigned to complement other primer sequences in a multiplex FL-PCR.

In another aspect of the present invention there is provided a kit for prenatal diagnosis, said kit comprising at least three (3) polymorphic microsatellite markers for use in a method of identifying a chromosome aneuploidy in a chromosome of a fetal cell, said method comprising:
obtaining a fetal cell;
identifying at least three polymorphic microsatellite markers on the chromosome; and
determining an allelic profile of the at least three (3) polymorphic microsatellite markers.

Preferably, the fetal cell is obtained from a cervical mucus sample as described herein.

In a preferred aspect, the kit further comprises a means to identify polymorphic microsatellite markers on specified chromosomes of fetal cells such that an allelic profile is obtained.

In an even further aspect, the kit includes a means to amplify the polymorphic microsatellite markers, preferably using PCR, more preferably FL-PCR. Means for amplifying the markers is described herein.

In an even further preferred aspect, there is provided a kit for diagnosing Down syndrome wherein the markers are selected from any of the markers for Down syndrome as described above.

In another aspect of the present invention there is provided a method of confirming fetal origin of a cell from a cervical mucus sample from an individual, said method including:
obtaining a fetal cell and a maternal cell from the same individual;
selecting at least three (3) polymorphic microsatellite markers characteristic of either the fetal or maternal cell; and
determining an allelic profile of the at least three (3) polymorphic microsatellite markers on the fetal cell and the maternal cell.

Preferably, the method of confirming fetal origin of a cell from an individual includes identifying a chromosome aneuploidy in a chromosome of the maternal cell and the fetal cell.

In addition, with the isolation of fetal cells from the cervix this non-invasive method can also detect mutations causing single gene disorders for example, Cystic Fibrosis.

It is also possible to combine the mutation detection for a single gene disorder with a DNA fingerprinting system and offer pregnant women diagnosis for both chromosomal aneuploidies and the indicated single gene disorder (refer to FIG. 3).

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

The present invention will now be more fully described with reference to the following examples. It should be understood, however, that the description following is illustrative only and should not be taken in any way as a restriction on the generality of the invention described above.

### EXAMPLES

### Example 1

### Diagnosis of Down Syndrome

(a) Collection and Preparation of Transcervical Cells

Collection of the transcervical cells from pregnant women during the first or second trimester is a similar procedure to a pap smear and involves direct aspiration of the cervical mucus, using a thin catheder (Aspiracath, Cook IVF), from the endocervical canal and the lower uterine pole. The tip of the catheder that contains the maternal mucus and transcervical cells is cut off into an eppendorf tube containing 0.5 ml of RPMI culture media and placed at 37° C. Gently the contents are mechanically removed from the inner tip of the catheder and suspended in culture media at 37° C.

(b) Identification and Isolation of Fetal Cells from Transcervical Samples

After collection, samples are treated with 0.5 ml of 20 mg/ml of N-acetyl-L-cysteine and further incubated with gentle shaking at 37° C. for 45 minutes. The entire sample is then washed twice in PBS before an incubation with 0.5 ml of enzyme cocktail (collagenases and proteases) for 1 hour at 37° C. During the incubation time disassociated cells are removed and remaining clumps of the original sample treated with fresh enzyme. After total disassociation the suspension of cells are then washed twice in PBS prior to immunofluorescent labeling.

A cocktail of three fetal specific antibodies (NDOG1, NDOG5 and FT1.41.1) that have been labeled with fluorescein is added to the cell suspension at 37° C. and allowed to bind specifically to the cell membranes of only fetal cells. NDOG1 stains the syncytiotrophoblasts, NDOG5 the syncytiotrophoblasts and cytotrophoblast cell columns and FT1.41.1 the syncytiotrophoblasts and villous cytotrophoblasts of first trimester pregnancy. None of these antibodies are reactive to maternal endometrium or cervical tissue. Under an inverted microscope using micromanipulation and microdissecting techniques with pulled glass pipettes, single and small clumps of fluorescently labeled fetal cells are identified, isolated and washed three times in PBS buffer before being transferred into 0.2 ml PCR tubes for analysis. As a negative control, maternal squamous cells are also isolated and washed three times in PBS buffer before being transferred into 0.2 ml PCR tubes for analysis.

(c) Confirmation of Fetal Origin and Trisomy 21 Diagnosis

This multiplex FL-PCR reaction incorporates five microsatellite markers found on chromosome 21. The allelic profile generated from this multiplex confirms either maternal or fetal origin as well as the presence or absence of chromosome 21. The FL-PCR reaction contains: 2.5 μl of 10×X PCR Buffer (500 mM KCl, 100 mM Tris-HCl, pH 9.0 and 15 mM MgCl₂), 0.5 μl of 10 mM dNTPs (200 μM), 0.3 μl of Taq polymerase (5 units/μl), 11.20 μl MQ-H₂O and 10.5 μl of primer mix making a final volume of 25 μl. Primer pairs include D21S1411, D21S11, D21S1413, D21S1442, and D21S1437 in each PCR reaction. All reaction mixes underwent manual “Hot Start” and multiplex FL-PCR was performed using the 9700 Thermocycler PCR machine (Applied Biosystems, Australia). Reactions were subjected to 35 thermal cycles consisting of denaturation for 45 seconds at 94° C., annealing for 45 seconds at 60° C., and extension for 1 minute at 72° C. With each single cell multiplex FL-PCR, positive and negative controls are included to ensure that PCR reaction mixed were functional and none of the reagents were contaminated.

Other examples include the simultaneous diagnosis of Down Syndrome and the common Cystic Fibrosis DeltaF508 mutation. The FL-PCR is as described above with the following changes: the primer mix contains four informative chromosome 21 microsatellite markers along with the primer pair for deltaF508 mutation detection.

(d) Readout of Fetal Origin and Diagnosis of Trisomy 21

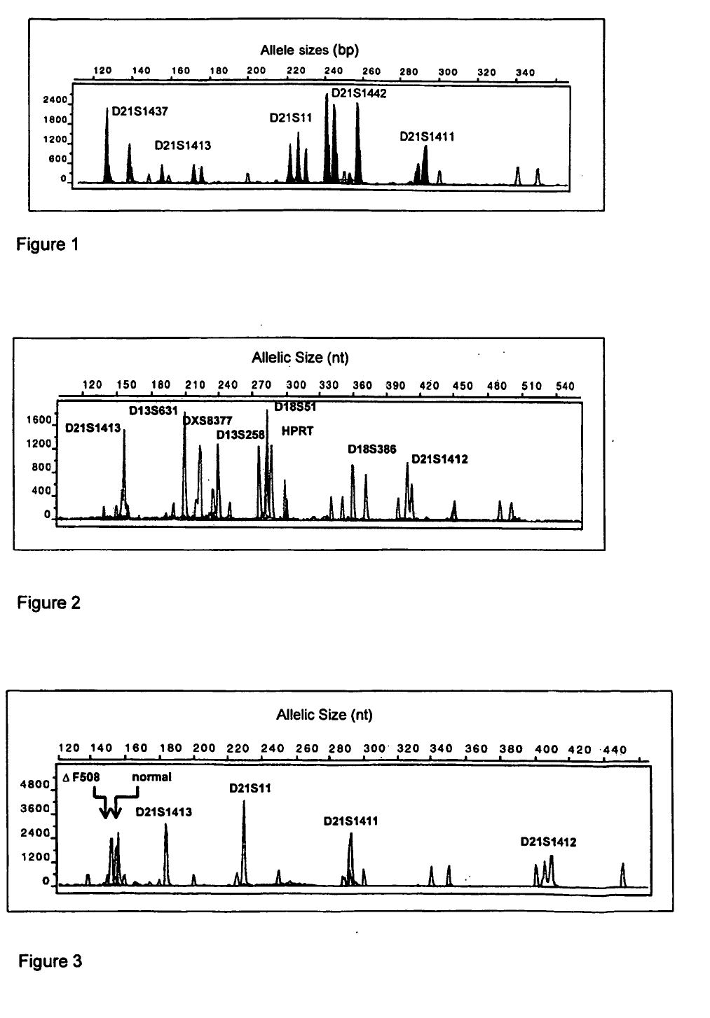

All FL-PCR products are analysed using the ABI Prism 3100 DNA Sequencer and associated Genescan 672 software (Applied Biosystems, Australia). Each PCR product (0.5-1.0 μl) is mixed with 9.75 μl of formamide and 0.25 μl of internal standard. Samples are denatured at 95° C. for 5 minutes, placed on ice and 10 μl loaded into a 96 well plate. Samples are subjected to electrokinetic injection and electrophoresed with automatic sizing by Genescan software. Genescan profiles or ‘fingerprints’ are generated showing the PCR products as coloured peaks dependent on the fluorescent dye used (see FIG. 1—a single cell allelic profile of a Down syndrome subject).

Microarrays can also be used to confirm fetal origin, diagnosis of single gene disorders and chromosomal abnormalities. On a single microarray it is possible to identify fetal cells using single nucleotide polymorphisms (SNP's), single gene disorders and chromosomal abnormalities. With this method the isolated and identified single fetal cell will firstly undergo whole genome amplification (WGA) by either PEP-PCR, DOP-PCR, linker adapter-PCR or MSDWGA. Fluorescently labeled product from WGA can be hybridised to the microarray platform and laser scanning of bound fluorescence will confirm fetal origin and diagnosis of chromosome aneuploidy of all 23 pairs of human chromosomes and identify any specific single gene defects.

### Example 2

### Diagnosis of Down Syndrome II

(a ) The collection and preparation of transcervical cells is followed as described above in example 1.

(b) The Identification and Isolation of Fetal Cells

After collection, samples are spread onto slides and fixed in 100% ethanol. Immunohistochemistry is performed using first trimester fetal specific antibodies to identify the fetal cells. The slides are then dehydrated. Laser capture microdissection technology is used to remove positively stained cells from the slide onto membranes that can be directly transferred into PCR tubes.

(c, d and e) FL-PCR DNA fingerprinting for trisomy 21 and the analysis of FL-PCR products and diagnosis of aneuploidy is followed as described above in example 1

### Example 3

### Simultaneous Diagnosis of Down Syndrome and Cystic Fibrosis DeltaF508 Mutation

(a & b) The collection and preparation of transcervical cells, and the identification and isolation of fetal cells is followed as described above in example 1.

(c) FL-PCR DNA Fingerprinting for Trisomy 21 and Cystic Fibrosis deltaF508 Diagnosis

The FL-PCR reaction is as described above with the following changes: the primer mix contains four informative chromosome 21 microsatellite markers along with the primer pair for deltaF508 mutation detection. Microsatellite markers outlined in Tables 2 & 3 are genotyped on parental genomic DNA to identify the heterozygous loci for incorporation into the DNA fingerprinting system. Final optimized primer pair concentrations are reaction and primer specific.

(d & e) Analysis of FL-PCR products and diagnosis of aneuploidy is as described above (see FIG. 3).

Finally it is to be understood that various other modifications and/or alterations may be made without departing from the spirit of the present invention as outlined herein.

TABLE 1
Details of aneuploid pregnancies

| | |
|---|---|
| Chromosome abnormalities | Chromosome abnormalities |
| in spontaneous abortions | in the newborn |

| | Incidence | | Incidence per |
|---|---|---|---|
| Abnormality | (%) | Abnormality | 10,000 births |
| Trisomy 13 | 2 | Trisomy 13 | 2 |
| Trisomy 16 | 15 | Trisomy 18 | 3 |
| Trisomy 18 | 3 | Trisomy 21 | 15 |
| Trisomy 21 | 5 | 45, X | 1 |
| Other trisomies | 25 | 47, XXX | 10 |
| Monosomy X | 20 | 47, XXY | 10 |
| Triploidy | 15 | 47, XYY | 10 |
| Tetraploidy | 5 | Unbalanced | 10 |
| | | rearrangements | |
| Other | 10 | Balanced | 30 |
| | | rearrangements | |

TABLE 2

Details of tetranucleotide microsatellite markers

| | | | | | |
|---|---|---|---|---|---|
| | | | | | Probability |
| Microsatellite | | Fluoro- | Allelic Size | Hetero- | of a tri-allelic |
| Marker | Primer sequence | chrome | Range | zygosity | pattern (%) |

| | | | | | | |
|---|---|---|---|---|---|---|
| D21S11 | F-tatgtgagtcaattccccaagtga | 6-FAM | 172-264 | bp | 0.93 | 72.5 |
| | R-gttgtattagtcaatgttctccag | | | | | |
| D21S1411 | F-atgatgaatgcatagatggatg | HEX | >239 | bp | 0.93 | 65.5 |
| | R-aatgtgtgtccttccaggc | | | | | |
| D21S1413 | F-ttgcagggaaaccacagtt | TET | <240 | bp | 0.88 | 62.5 |
| | R-tccttggaataaattcccgg | | | | | |
| D21S1437 | F-tgaacatacatgtacatgtgtctgg | 6-FAM | 107-143 | bp | 0.93 | 65.5 |
| | R-ttctctacatatttactgccaacac | | | | | |
| D21S1442 | F-cactgcagacggcatgaacttcc | TET | 237-261 | bp | 0.80 | 67.5 |
| | R-ccagaatcacatgagccaattcc | | | | | |

TABLE 3
Tetranucleotide microsatellite markers identified for DNA fingerprinting
and aneuploidy diagnosis of chromosomes 13, 18, 21, X and Y

| Chromosome | Microsatellite Marker |
|---|---|
| Chromosome 21 | D21S1412 |
| | D21S1414 |
| | D21S1435 |
| | D21S1808 |
| | D21S1270 |
| Chromosome 13 | D13S631 |
| | D13S258 |
| | D13S634 |
| | D13S317 |
| | D13S800 |
| X Chromosome | DXS8377 |
| | HUMARC |
| | HPRT |
| | DXS1283E |
| | SBMA |
| | X22 |
| Y Chromosome | DYS391 |
| | DYS393 |
| | DYS390 |
| Chromosome 18 | D18S535 |
| | D18S51 |
| | MBP |
| | D18S978 |
| | D18S1002 |
| | D18S974 |
| | D18S849 |
| | D18S865 |
| | D18S877 |
| | D18S386 |

1-40. (canceled)

41. A method of retrieving cells from a cervical mucus sample, comprising:
treating a cervical mucus sample with one or more agents selected from the group consisting of a collagenase, a protease, a liberase blendzyme, and a mucolytic agent, to disassociate cells from the cervical mucus sample; and
retrieving disassociated cells from the sample.

42. A method according to claim 41 wherein the cervical mucus sample is selected from the group consisting of
(i) a transcervical mucus sample,
(ii) a cervical mucus sample that is obtained from at least one cervical region selected from endocervical canal and lower uterine pole,
(iii) a cervical mucus sample obtained during a first or second trimester of pregnancy.

43. The method of claim 41 wherein the cervical mucus sample is treated with a collagenase, a protease and a liberase blendzyme.

44. The method of claim 41 wherein the cervical mucus sample is treated with (i) a collagenase, a protease and a mucolytic agent, or (ii) a collagenase, a protease, a liberase blendzyme and a mucolytic agent.

45. The method of claim 41 or claim 44 wherein the cervical mucus sample is treated with a mucolytic agent prior to treatment with a collagenase and a protease.

46. A disassociated cell prepared by a method according to any one of claims 41 to 45.

47. A method of retrieving a fetal cell from a cervical mucus sample, said method comprising:
treating a cervical mucus sample with one or more agents selected from the group consisting of a collagenase, a protease, a liberase blendzyme, and a mucolytic agent, to disassociate cells from the cervical mucus sample;
subjecting the cells to a fetal antibody;
identifying cells that have bound to the fetal antibody; and
retrieving the identified cells.

48. A method of identifying a fetal cell, said method comprising:
treating a cervical mucus sample with one or more agents selected from the group consisting of a collagenase, a protease, a liberase blendzyme, and a mucolytic agent, to disassociate cells from the cervical mucus sample;
subjecting the cells to a fetal antibody;
identifying cells that have bound to the fetal antibody.

49. A method according to either claim 47 or claim 48 wherein the fetal antibody is a first trimester fetal specific antibody.

50. A method according to either claim 47 or claim 48 wherein the fetal antibody is used singularly or in combination with another antibody to identify the fetal cells.

51. A method according to claim 50 wherein one or more antibodies are used to identify fetal cells.

52. The method of either claim 47 or claim 48 comprising comparing at least one microsatellite marker in the fetal cell to said microsatellite marker in a maternal control cell, wherein said fetal cell shares a microsatellite marker with the maternal cell.

53. A method according to claim 52 wherein the step of comparing comprises identifying a microsatellite marker with a forward primer sequence selected from the group consisting of:

| | |
|---|---|
| tatgtgagtcaattccccaagtga; | (SEQ ID NO: 1) |
| atgatgaatgcatagatggatg; | (SEQ ID NO: 2) |
| ttgcagggaaaccacagtt; | (SEQ ID NO: 3) |
| tgaacatacatgtacatgtgtctgg; | (SEQ ID NO: 4) |
| and | |
| cactgcagacggcatgaacttc. | (SEQ ID NO: 5) |

54. A method according to claim 53 wherein the microsatellite marker is identified with a reverse primer sequence selected from the group consisting of:

| | |
|---|---|
| gttgtattagtcaatgttctccag; | (SEQ ID NO: 6) |
| aatgtgtgtccttccaggc; | (SEQ ID NO: 7) |
| tccttggaataaattcccgg; | (SEQ ID NO: 8) |
| ttctctacatatttactgccaacac; | (SEQ ID NO: 9) |
| and | |
| ccagaatcacatgagccaattcc. | (SEO ID NO: 10) |

55. A fetal cell prepared by a method according to claim 47.

56. A method of identifying a fetal cell, said method comprising:
treating a cell sample with one or a plurality of antibodies selected from the group consisting of NDOG1, NDOG5 and FT1.41.1 or an equivalent thereof; and
identifying a cell that has bound to the antibody.

57. A method according to claim 56 comprising treating the sample with a plurality of antibodies.

58. A composition for identifying fetal cells, said composition comprising antibodies NDOG1, NDOG5 and FT1.41.1.

59. A method of characterizing a fetal cell, said method comprising:
(i) retrieving a fetal cell from a cervical mucus sample according to claim 47;
(ii) subjecting the fetal cell to a procedure selected from the group consisting of:
(a) multiplex FL-PCR;
(b) WGA, hybridisation and microarray analysis; and
(c) extraction of mRNA, cDNA libraries, hybridisation and gene expression microarray analysis; and
(iii) analysing results of the procedure to characterize the cell.

60. A method according to claim 59 wherein the fetal cell is characterised for biochemical, metabolic or genetic disorders.

61. A method of identifying a chromosome aneuploidy in a chromosome of a fetal cell, said method comprising:
obtaining a fetal cell comprising a chromosome from a cervical mucus sample prepared by a method according to claim 47;
identifying at least three polymorphic microsatellite markers on the chromosome; and
determining an allelic profile of at least three polymorphic microsatellite markers.

62. A method according to claim 61 wherein the allelic profile is determined by one or more of five microsatellite markers.

63. A method of prenatal diagnosis, said method comprising:
obtaining a fetal cell that comprises a chromosome from a cervical mucus sample prepared by a method according to claim 47;
identifying at least three polymorphic microsatellite markers on the chromosome;
determining an allelic profile of the at least three polymorphic microsatellite markers; and
correlating the allelic profile with a condition for prenatal diagnosis.

64. A method according to claim 63 wherein prenatal diagnosis comprises determining the presence of a genetic mutation in a fetal cell and wherein the genetic mutation is selected from the group consisting of a chromosomal aneuploidy, a point mutation, a translocation, a trinucleotide repeat expansion, an insertions and a deletion.

65. A method according to claim 64 wherein the genetic mutation is associated with a condition selected from the group consisting of cystic fibrosis, beta-thalassaemia, Huntington's Disease, Fragile X, Myotonic Dystrophy, Duchenne Muscular Dystrophy, Sickle Cell Anaemia, Turners syndrome (XO), Kinefelter's syndrome (XXY), XXX females and XYY males, Triploidy (69, XXX or XXY or XYY), Patau's syndrome (trisomy 13) and Edward's syndrome (trisomy 18), and Down syndrome (trisomy 21).

66. A method according to claim 65 wherein the chromosome aneuploidy occurs in a human chromosome selected from the group consisting of chromosomes 21, 18, 13, X and Y.

67. A method according to claim 66 wherein the allelic profile indicates a trisomy of chromosome 21.

68. A method according to claim 64 wherein the genetic mutation is associated with Down Syndrome.

69. A method according to either claim 61 or claim 63 wherein the microsatellite marker is identified with a forward primer sequence selected from the group consisting of:

| | |
|---|---|
| tatgtgagtcaattccccaagtga; | (SEQ ID NO: 1) |
| atgatgaatgcatagatggatg; | (SEQ ID NO: 2) |
| ttgcagggaaaccacagtt; | (SEQ ID NO: 3) |
| tgaacatacatgtacatgtgtctgg; | (SEQ ID NO: 4) |
| and | |
| cactgcagacggcatgaacttc. | (SEO ID NO: 5) |

70. A method according to either claim 61 or claim 63 wherein the microsatellite marker is identified with a reverse primer sequence selected from the group consisting of:

| | |
|---|---|
| gttgtattagtcaatgttctccag; | (SEQ ID NO: 6) |
| aatgtgtgtccttccaggc; | (SEQ ID NO: 7) |
| tccttggaataaattcccgg; | (SEQ ID NO: 8) |
| ttctctacatatttactgccaacac; | (SEQ ID NO: 9) |
| and | |
| ccagaatcacatgagccaattcc. | (SEQ ID NO: 10) |

71. A method according to either claim 61 or claim 63 wherein the microsatellite marker comprises a marker selected from the group consisting of markers in Table 2.

72. A method of confirming fetal origin of a cell from an individual, said method comprising:
obtaining from a pregnant woman (i) a fetal cell from a cervical mucus sample by a method according to claim 47, and (ii) a maternal cell from the same woman;
selecting at least three polymorphic microsatellite markers that are characteristic of either the fetal cell or the maternal cell; and
determining an allelic profile of the at least three polymorphic microsatellite markers of the fetal cell and of the maternal cell.

73. A method according to claim 72 comprising confirming fetal origin of a cell by identifying a chromosome aneuploidy in a chromosome of the maternal cell and of the fetal cell.

74. (Previously Presented) A method of detecting a single gene disorder, said method comprising:
obtaining a fetal cell from a cervical mucus sample prepared by a method according to claim 47; and
detecting a mutation in a gene of the fetal cell.

75. A method according to claim 74 wherein the single gene disorder is selected from the group consisting of cystic fibrosis, beta-thalassaemia, Huntington's Disease, Fragile X, Myotonic Dystrophy, Duchenne Muscular Dystrophy, and Sickle Cell Anaemia.

76. A method according to claim 74 wherein the genetic disorder is cystic fibrosis.
